(19) United States
(12) Patent Application Publication
(10) Pub. No.: US 2026/0120388 A1
(43) Pub. Date: Apr. 30, 2026

(54) LATENT DIFFUSION-ENABLED SYSTEM FOR EXTENDING THE FIELD OF VIEW (FOV) OF COMPUTED TOMOGRAPHY (CT) IMAGES

(71) Applicant: VANDERBILT UNIVERSITY, Nashville, TN (US)

(72) Inventors: Lianrui ZUO, Nashville, TN (US); Bennett A. LANDMAN, Nashville, TN (US)

(21) Appl. No.: 19/374,751

(22) Filed: Oct. 30, 2025

Related U.S. Application Data
(60) Provisional application No. 63/714,598, filed on Oct. 31, 2024.

(51) Int. Cl. G06T 15/08; G06T 5/60; G06T 5/70

(52) U.S. Cl. CPC G06T 15/08; G06T 5/60; G06T 5/70; G06T 2207/10081; G06T 2207/20081; G06T 2207/20084; G06T 2207/30056; G06T 2207/30061; G06T 2207/30084; G06T 2210/41

(57) ABSTRACT

A system and method for extending the field of view (FOV) of input computed tomography (CT) images by using a trained latent diffusion model (LDM) to synthesize additional CT images beyond the field of view of the captured input CT images. The system encodes two-dimensional CT image slices into latent representations, which are then used to form three-dimensional contexts for training the latent diffusion model to capture complex anatomical structures and inherent inter-organ relationships as prior knowledge. Leveraging those learned inter-organ relationships, the disclosed system synthesizes additional CT image slices by performing a guided reverse diffusion process in which latent representations of known input CT images are used to correct the synthesis at each step, enabling the system to generate additional anatomically coherent CT images in a zero-shot manner.

### CROSS-REFERENCE TO RELATED APPLICATIONS

This application claims priority to U.S. Prov. Pat. Appl. No. 63/714,598, filed Oct. 31, 2024, which is hereby incorporated by reference in its entirety.

### FEDERAL FUNDING

This invention was made with government support under Grant Numbers CA253923 and CA275015 awarded by the National Institutes of Health (NIH). The government has certain rights in the invention.

### BACKGROUND

The human lungs play an important role in respiration and maintaining overall physiological homeostasis. However, the impact of lung diseases often extends beyond the respiratory system, affecting other organs such as the liver and the kidneys. For instance, chronic obstructive pulmonary disease (COPD) can lead to hepatic congestion, while pulmonary complications are common in patients with chronic kidney disease (CKD). Extensive research has been conducted to study the interconnection between the human lung and other organs, emphasizing the importance of viewing the human body as an integrated system. Understanding these interconnections is crucial for comprehensive patient care, treatment planning, and monitoring disease progression.

To minimize radiation dose and cost, however, clinical and research chest CT exams are typically focused solely on the lungs, hindering the ability to provide comprehensive analysis and gain insights into the impact of lung diseases on other organs. The National Lung Screening Trail (NLST), for example, recommends a computed tomography (CT) scanning protocol that exclusively covers the lung region.

Accordingly, there is a need for a system and method that extends the field of view of chest CT images in the Z direction to enable clinicians and researchers to evaluate the health of other organs.

Ideally, a machine learning-enabled system for extending the field of view of CT images would be trained using large field of view datasets, such as whole-body CT images. However, such comprehensive datasets are not always available, and even when they are, their quantity is often limited.

Xu et al.¹ describe a system for extending the field of view of axial chest CT image slices in the axial plane (to fill in the missing subcutaneous fat due to truncation) using generative AI technology. Extending the field of view of axial CT image slices in a longitudinal direction, however, is a more challenging technical problem, in part because of the limited availability of large field of view datasets. Additionally, the methods described by Xu et al. require significant computational resources. ¹Xu, K., Khan, M. S., Li, T. Z., Gao, R., Terry, J. G., Huo, Y., Lasko, T. A., Carr, J. J., Maldonado, F., Landman, B. A., et al., “AI body composition in lung cancer screening: added value beyond lung cancer detection,” Radiology 308(1), e222937 (2023); Xu, K., Li, T., Khan, M. S., Gao, R., Antic, S. L., Huo, Y., Sandler, K. L., Maldonado, F., and Landman, B. A., “Body composition assessment with limited field-of-view computed tomography: A semantic image extension perspective,” Medical Image Analysis 88, 102852 (2023).

### SUMMARY

In order to overcome those and other disadvantages of the prior art, the disclosed system extends the field of view of input CT images (e.g., chest CT images) by using a trained latent diffusion model (LDM) to synthesize additional CT images (e.g., abdominal CT images) beyond the field of view of the captured input CT images. The system encodes two-dimensional CT image slices into latent representations, which are then used to form three-dimensional contexts for training the latent diffusion model to capture complex anatomical structures and inherent inter-organ relationships as prior knowledge. Leveraging those learned inter-organ relationships, the disclosed system synthesizes additional CT image slices by performing a guided reverse diffusion process in which latent representations of known input CT images are used to correct the synthesis at each step, enabling the system to generate additional anatomically coherent CT images in a zero-shot manner.

The latent diffusion model may be trained using two partial datasets (e.g., chest CT images and abdominal CT images) having overlapping regions. While each of those partial datasets is focused primarily on its respective organs with limited fields of view, the overlapping regions that serve as a “bridge” and allow the latent diffusion model to capture the inter-organ relationship (e.g., across the lungs, liver, and the kidneys) during training. Accordingly, the disclosed system eliminates the need for datasets with large fields of view, which have limited availability.

By using a latent diffusion model to transform the three-dimensional CT images into a two-dimensional problem, the disclosed system reduces the computation burden while maintaining three-dimensional context information of the human anatomy. Additionally, in embodiments realized using a variational autoencoder, the disclosed system captures latent representations that are indicative of the smooth transitions across the human body (and, by extension, the smooth transitions among and across CT image slices).

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1A illustrates the estimated field of view of the National Lung Screening Trail (NLST) dataset.

FIG. 1B illustrates an example of an extended field of view provided by embodiments of a disclosed Spatial Coverage Optimization with Prior Encoding (SCOPE) system.

FIG. 2 is a diagram illustrating the SCOPE system according to exemplary embodiments.

FIG. 3 is a diagram illustrating a first phase of a training process used to train exemplary embodiments of the SCOPE system.

FIG. 4 is a diagram illustrating a second phase of the training process used to train exemplary embodiments of the SCOPE system.

FIG. 5 is a diagram illustrating a zero-shot field of view (FOV) extension process performed by exemplary embodiments of the SCOPE system.

FIG. 6 is a diagram illustrating an uncertainty quantification process performed by exemplary embodiments of the SCOPE system.

FIG. 7 are example images demonstrating the ability of the SCOPE system to extend the field of view of input CT images.

FIG. 8 are graphs illustrating volumetric agreement of the liver and kidneys between acquired ground truth images and synthetically extended images generated by embodiments of the SCOPE system.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

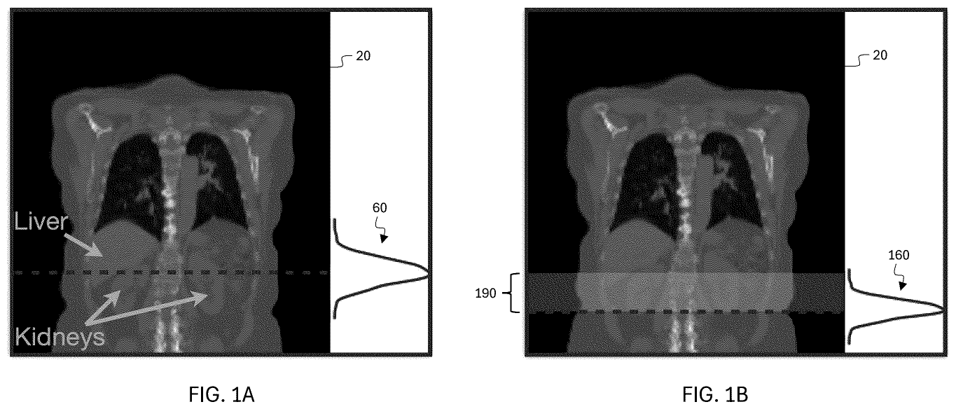

Most lung screening protocols focus solely on the lung region. FIG. 1A, for example, illustrates an estimated field of view (FOV) distribution 60 of the vertical position of the lowest slice of each three-dimensional computed tomography (CT) scan in the National Lung Screening Trail (NLST) dataset relative to a reference CT image 20. While the lung region is covered in all NLST data, liver and kidney regions are only partially covered in the NLST dataset as shown in FIG. 1A.

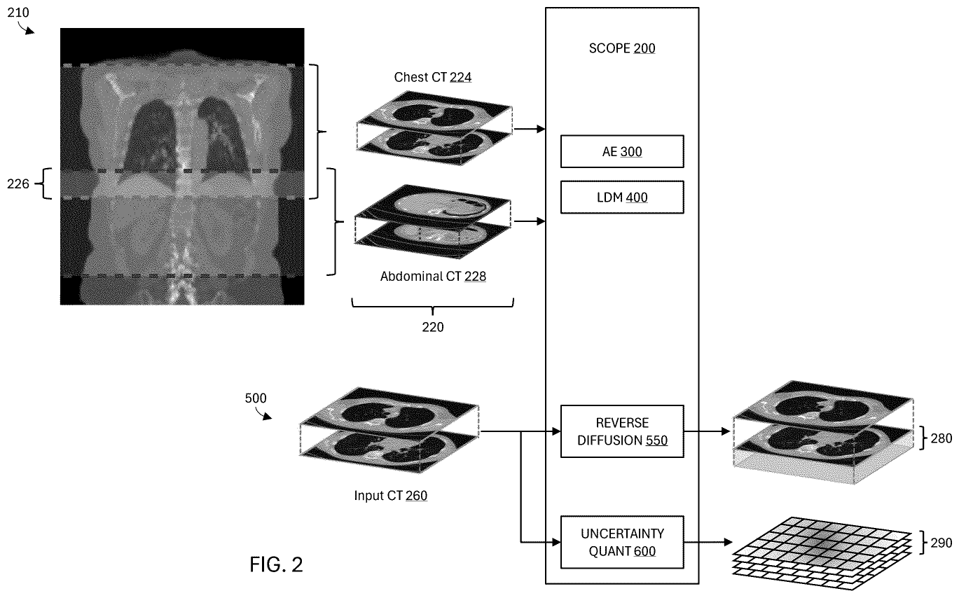

Because of the significance of inter-organ relationships described above, a Spatial Coverage Optimization with Prior Encoding (SCOPE) system 200 is described below with reference to FIGS. 2-7 that extends the FOV of captured CT images. As shown in FIG. 1B, for example, FOV extension using the SCOPE system 200 can extend the field of view of the NLST dataset beyond the lung region and into the shaded area 190 covering the liver and kidney regions. Accordingly, the estimated FOV distribution 160 of the vertical position of the lowest slice of each three-dimensional CT scan in the NLST dataset after FOV extension by the disclosed SCOPE system is lower, relative to the reference computed CT image 20, than the original FOV distribution 60 of the NLST dataset.

FIG. 2 is a diagram illustrating a training process 210 for training the SCOPE system 200 and a zero-shot field of view (FOV) extension process 500 performed by the SCOPE system 200 according to exemplary embodiments.

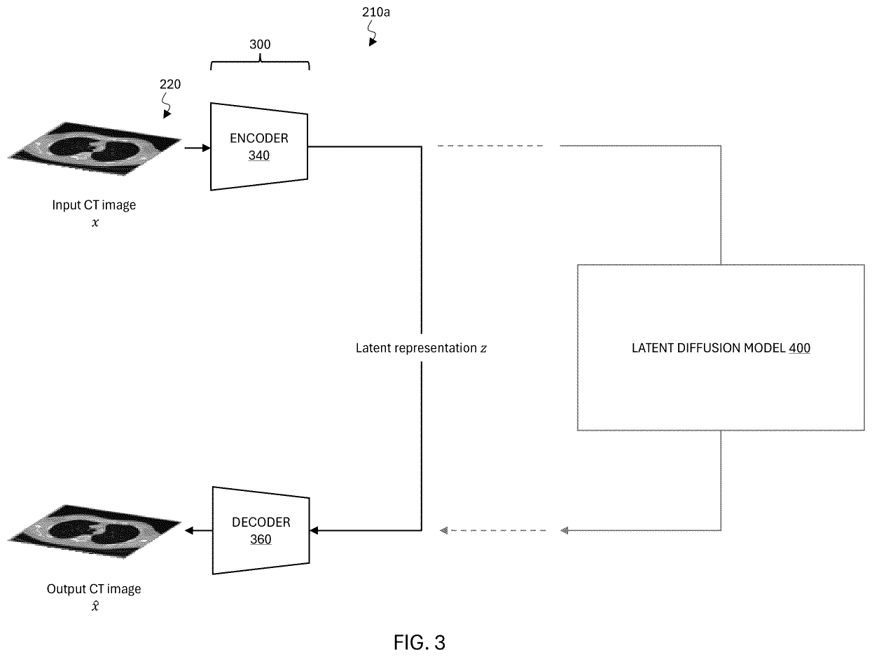

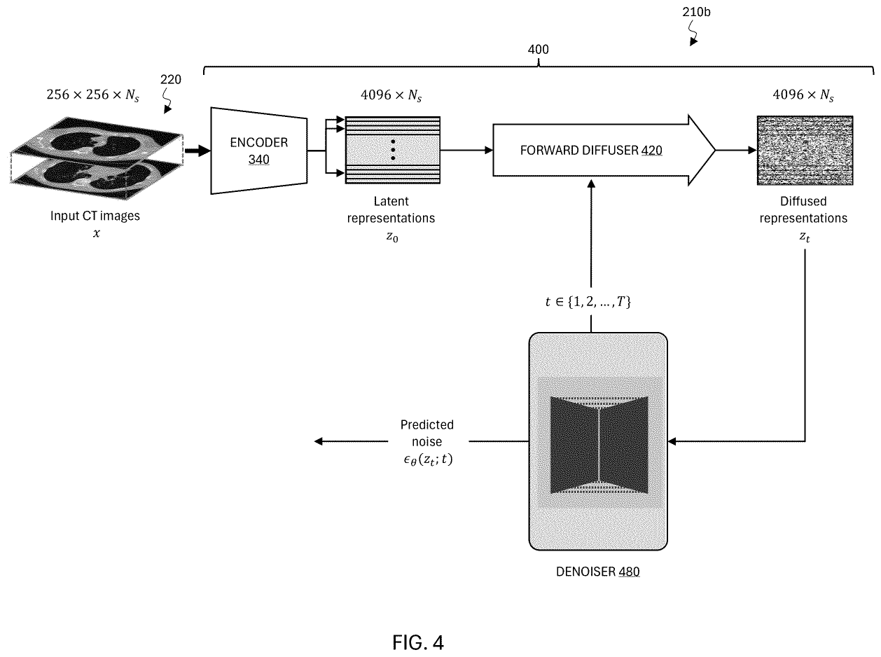

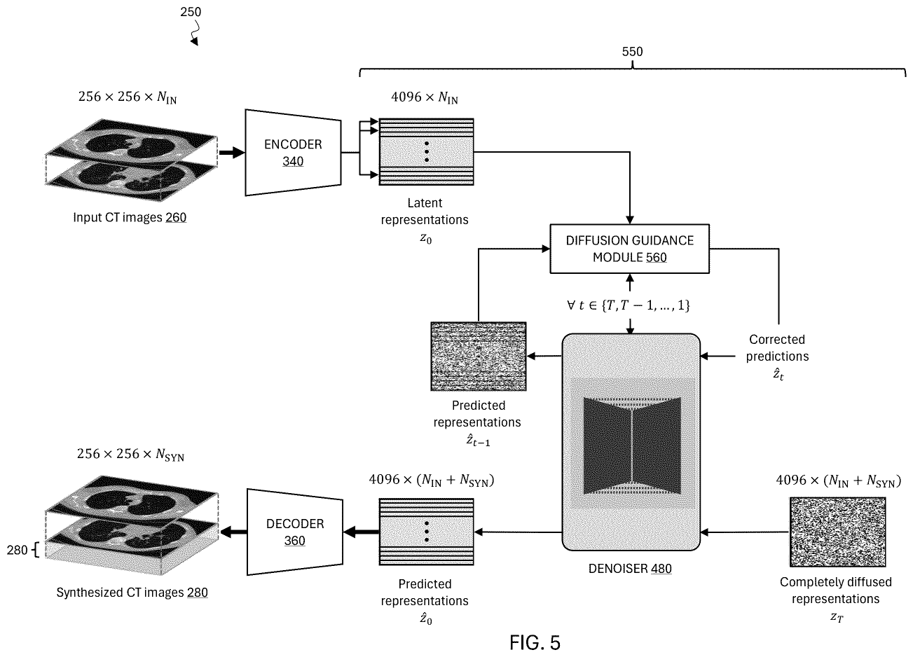

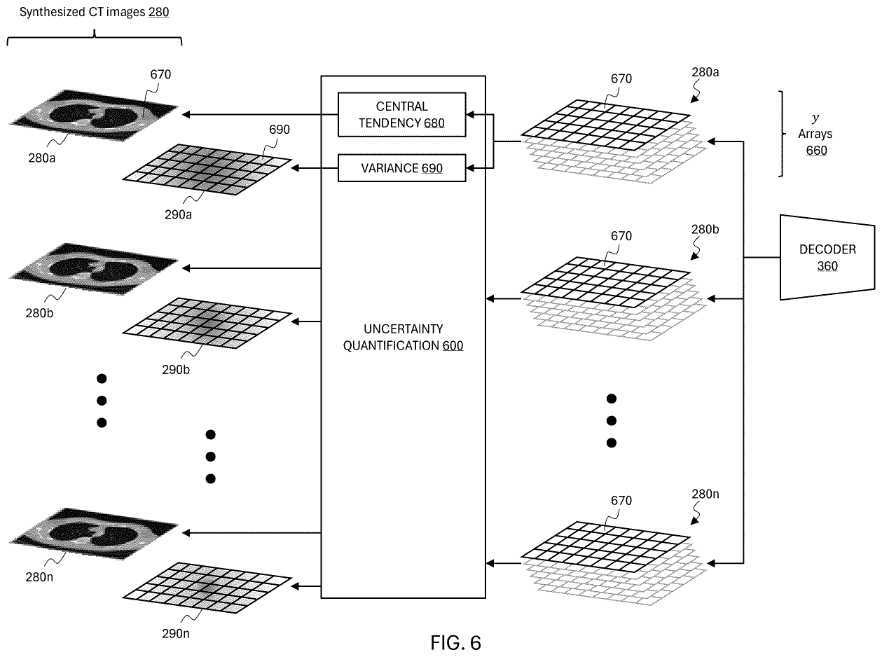

In the embodiment of FIG. 2, the SCOPE system 200 includes an autoencoder 300 (described in detail below with reference to FIG. 3) and a latent diffusion model 400 (described in detail below with reference to FIG. 4). In the example training process 210 of FIG. 2, which is described in detail below with reference to FIGS. 3 and 4, the SCOPE system 200 is trained using training data 220 that includes chest CT images 224 and abdominal CT images 228 having an overlapping field of view 226. Once trained on the training data 220, the SCOPE system 200 is configured to perform the zero-shot FOV extension process 500 to extend the field of view of input CT images 260, for example by performing a reverse diffusion process 550 (described in detail below with reference to FIG. 5) to generate synthesized CT images 280 outside of the field of view of the input CT images 260 and performing an uncertainty quantification process 600 (described in detail below with reference to FIG. 6) to generate confidence maps 290 indicative of the predicted accuracy of each pixel value in each synthesized CT image 280.

FIG. 3 is a diagram illustrating a first phase 210a of the training process 210 used to train the SCOPE system 200 according to exemplary embodiments.

As shown in FIG. 3, the autoencoder 300 includes an encoder 340 and a decoder 360. (In preferred embodiments, the autoencoder 300 is a variational autoencoder, which provides important technical benefits relative to other autoencoders as described below.) The autoencoder 300 is trained on the training data 220 to reduce the dimensionality of each input CT image x by generating a lower-dimensional latent representation z indicative of the input CT image x. In the embodiment of FIG. 3, for example, the autoencoder 300 is trained to map each two-dimensional axial slice in the training data 220 (input CT image x) to a 4096-dimensional vector (latent representation z) in a 4096-dimensional latent space.

As shown in FIG. 3, the encoder 340 generates a latent representation z of each input CT image x and the decoder 360 synthesizes an output CT image \hat{x} based on the latent representation z. By training the encoder 340 and the decoder 360 to minimize the difference between the output CT image \hat{x} and the input CT image x from which it was generated, the encoder 340 is trained to generate latent representation z that preserve the features necessary for the decoder 360 to reconstruct the input CT images x. In embodiments wherein the autoencoder 300 is a variational autoencoder, for example, the training objective may be to minimize the VAE loss [symbol] _{VAE} as defined as:

$${ℒ}_{VAE} = {‖ \hat{x} - x ‖}_{1} + λ {𝒟}_{KL} [ p ( z \vert x ) \vert \vert p ( z ) ] \qquad [ Eq . 1 ]$$

where [symbol] _{KL} is the Kullback-Leibler divergence, p(z) is the standard Gaussian distribution [symbol] (0, I), λ is a hyperparameter.

After the autoencoder 300 is trained in the first phase 210a, the latent diffusion model 400 is trained as described below.

FIG. 4 is a diagram illustrating a second phase 210b of the training process 210 used to train the SCOPE system 200 according to exemplary embodiments.

As shown in FIG. 4, the latent diffusion model 400 includes a forward diffuser 420 and a neural network denoiser 480. The latent representations z generated from consecutive CT slices (input CT images x) are stacked to form a three-dimensional context (latent representations z₀). At each of T steps t, the forward diffuser 420 is configured to inject a predetermined level of noise E into the latent representations to form diffused representations zₜ. In other words, the initial latent representations z₀ output by the encoder 340 step 0 (t=0) do not include any noise ϵ injected by the forward diffuser 420 while the final latent representations z_{T} at step T (t=T) do not include any of the original signal and are instead entirely noise E injected by the forward diffuser 420. More formally, the forward diffusion process performed by the forward diffuser 420 may be defined as:

$$q ( {z}^{t} \vert {z}_{t - 1} ) = 𝒩 ( {z}_{t} ; \sqrt{1 - {β}_{t}} {z}_{t - 1} , {β}_{t} I ) \qquad [ Eq . 2 ]$$

where βₜ controls the level of noise being injected at each step t∈{1, 2, . . . T}.

The neural network denoiser 480 is trained to predict the noise E injected by the forward diffuser 420. More specifically, the neural network denoiser 480 is trained on the stacked latent representations z generated from the training data 220 to minimize the difference between the predicted noise ϵ_{θ}(zₜ; t) output by the neural network denoiser 480 and the actual noise ϵ injected by the forward diffuser 420. More formally, the training objective for training the neural network denoiser 480 may be defined as minimizing the latent diffusion model loss [symbol] _{DM} as follows:

$${ℒ}_{LDM} = {‖ ϵ - {ϵ}_{θ} ( {z}_{t} ; t ) ‖}_{2}^{2} \qquad [ Eq . 3 ]$$

where ϵ˜ [symbol] (0, I) is the Gaussian noise and t is a randomly sampled time step between 0 and T.

By training the latent diffusion model 400 on the stacked latent representations z generated from the training data 220, the SCOPE system 200 learns to model the complex anatomical structures and relationships in the latent space z, capturing the prior anatomical knowledge of the human body. In the zero-shot FOV extension process 500 described below with reference to FIG. 5, those learned anatomical relationships enable the SCOPE system 200 to infer missing information based on the latent representations z of available input CT images 260 and use that inferred information to synthesize additional CT images 280, expanding the field of view of the available input CT images 260.

The autoencoder 300 and the neural network denoiser 480 require input data of a fixed dimension. Meanwhile, the number of slices S in each three-dimensional image in the training data 220 may vary. Accordingly, the SCOPE system 200 may be trained using randomly selected segments of Nₛ consecutive slices. For example, the SCOPE system 200 may be trained using randomly selected segments of 64 consecutive slices having a slice thickness of 3 mm without a slice gap. In those embodiments, each sample provided to the SCOPE system 200 covers approximately 20 cm of the human body, thereby providing three-dimensional context for the latent diffusion model 400 to capture.

FIG. 5 is a diagram illustrating the zero-shot FOV extension process 500 performed by the SCOPE system 200 according to exemplary embodiments.

As shown in FIG. 5, the SCOPE system 200 receives input CT images 260 and generates synthesized CT images 280 that expand the field of view of those input CT images 260. For example, the input CT images 260 may be chest CT images and the synthesized CT images 280 may be abdominal CT images inferred by the SCOPE system 200 based on the received chest CT images and the complex anatomical structures and relationships learned during the training process 210 described above.

During the FOV extension process 500, the encoder 340 encodes the input CT images 260 to form a stack of latent representations z₀ as described above and, beginning with completely diffused representations z_{T} at step T, the SCOPE system 200 performs a guided reverse diffusion process 550. At each step t (from T down to 1) the neural network denoiser 480 predicts the noise ϵθ(zₜ; t) present in the latent representations zₜ for the current step t, which is subtracted from the predicted representations \hat{z}ₜ at step t to form the predicted representations \hat{z}ₜ₋₁ for the following step t−1. Critically, the reverse diffusion process 550 performed by the SCOPE system 200 is guided by a diffusion guidance module 560 that uses the latent representations z₀ of the input CT images 260 to correct the predicted representations 2 output by the neural network denoiser 480. More formally, before the neural network denoiser 480 generates the predicted representations \hat{z}ₜ₋₁ at each step t, the diffusion guidance module 560 modifies the predicted representations \hat{z}ₜ generated by the neural network denoiser 480 in the previous step t+1 by incorporating the real z₀ values, to which the mathematically appropriate level of noise ϵ for the corresponding step t has been applied, as follows:

$${\hat{z}}_{t} ← {z}_{t} ∘ ℳ + {\hat{z}}_{t} ∘ ( 1 - ℳ ) \qquad [ Eq . 4 ]$$

where \hat{z}ₜ on the left side of the equation represents the corrected latent representations output by the diffusion guidance module 560 and is provided as input to the neural network denoiser 480 at step t, zₜ represents the real latent representations z₀ of the input CT images 260 with a mathematically appropriate level of noise E for the corresponding step t applied, \hat{z}ₜ on the right side of the equation represent the initial, uncorrected representations predicted by the neural network denoiser 480 at step t+1, [symbol] is an array having the same dimension as z and values of 1 for positions corresponding to acquired slices (in the input CT images 260) and 0 for positions corresponding to unavailable slices (to be synthesized as synthesized CT images 280); and the “∘” operator indicates element-wise multiplication.

At the conclusion of the guided reverse diffusion process (step 0), the neural network denoiser 480 outputs a final, complete stack of predicted representations \hat{z}₀. To generate N_{SYN} synthesized CT images 280 based on N_{IN} input CT images 260, the neural network denoiser 480 generates N_{SYN}+N_{IN} predicted representations \hat{z}₀ based on the N_{IN} latent representations z₀ generated by the encoder 340. Those N_{SYN}+N_{IN} predicted representations \hat{z}₀ include the N_{IN} latent representations z₀ generated by the encoder 340 and N_{SYN} additional predicted representations \hat{z}₀. The decoder 360 then takes only the N_{SYN} newly generated representations \hat{z}₀ from that stack (corresponding to the unavailable slices) to generate N_{SYN} synthesized CT images 280.

By training the latent diffusion model 400 to model generally-applicable anatomical structures and inter-organ relationships and then applying that generally-applicable information to the new task of generating synthetic CT images 280 during the FOV extension process 500, the disclosed SCOPE system 200 can generate those synthetic CT images 280 in a “zero-shot” manner (i.e., without having to perform the time consuming and computationally-expensive guided reverse diffusion process 550 during the training process 210). In other words, the disclosed FOV extension process 500 (when performed by the disclosed SCOPE system 200 having been trained in accordance with the training process 210 described above) eliminates the need to, for example, mask out portions of the training data 220 and train a model to regenerate the masked out portions of the training data.

FIG. 6 is a diagram illustrating an uncertainty quantification process 600 performed by exemplary embodiments of the SCOPE system 200.

As described above with reference to FIG. 5, the scope system 200 extends the field of view of a three-dimensional volume of N_{IN} input CT images 260 by generating synthesized CT images 280a, 280b, . . . , 280n, which collectively form a three-dimensional volume of N_{SYN} synthesized CT images 280. As shown in FIG. 6, each synthesized CT image 280 may be realized as an array 660 of pixel values 670 (e.g., a 256×256 array of pixel values 670). To quantify the confidence in the predicted accuracy of each of the predicted pixel values 670 in each of the synthesized CT images 280, the SCOPE system 200 may perform the guided reverse diffusion process 550 multiple times to generate y arrays 660 corresponding to each synthesized CT image 280 and calculate the pixel level variance 690 at each pixel location of the y pixel values 670 across the y arrays 660.

To generate each synthesized CT image 280, the SCOPE system 200 may select any of the y pixel values 670 (from any of the y arrays 660) at each pixel location. Alternatively, the SCOPE system 200 may generate each synthesized CT image 280 by calculating a measure of central tendency 680 of the y pixel values 670 (e.g., the mean, the median, or the mode) at each pixel location across all of the y arrays 660 generated for that synthesized CT image 280. Using the mean pixel value 670 as the measure of central tendency 680 provides a true composite of all of the y pixel values 670 and, by using all of the information from all of the many samples, may act as a “smoothing filter” that averages out minor, high-frequency noise and generates a smooth, “softer” looking synthesized CT image 280. The mean pixel value 670, however, may be highly sensitive to outliers, which may be an issue in stochastic generative models (like the guided reverse diffusion process 550) that can sometimes produce artifacts (e.g., a CT image 280 with a bright white or dark black patch). If even one or two arrays 660 in a sample of 100 arrays 660 have a wildly incorrect pixel value 670, those will significantly pull the mean in that direction. Selecting the median pixel value 670, by contrast, ignores those artifacts. Accordingly, in some embodiments the measure of central tendency 680 used to calculate each pixel value 670 at each pixel location in each synthesized CT image 280 may be the median pixel value 670 across all of the y arrays 660.

When performing a generative modeling process, prediction variance serves as a powerful and direct measure of model uncertainty. Accordingly, the pixel level variance 690 at each pixel location of each synthesized CT image 280 may form a confidence map 290 quantifying the uncertainty of each pixel value 670 at each pixel location. When the same guided reverse diffusion process 550 is run multiple times to synthesize a predicted pixel value 670, high variance 690 across those multiples runs indicates a lack of model consensus (that the latent diffusion model 400 is in a sense “unsure” of the correct prediction), decreasing confidence in the accuracy of that predicted pixel value 670. Conversely, if the latent diffusion model 400 repeatedly converges on the same pixel value 670 (or very similar pixel values 670), that low variance 690 implies a strong, stable solution and increases the confidence in the accuracy of the pixel value 670.

In some embodiments, the SCOPE system 200 may output all N_{SYN} synthesized CT images 280 along with N_{SYN} confidence maps 290 indicative of the predicted accuracy of each pixel value 670 in the corresponding synthesized CT image 280. In other embodiments, the SCOPE system 200 may output only the CT images 280 (or only the pixel values 670) having less than a predetermined level of variance 690.

In preferred embodiments, the autoencoder 300 may be a variational autoencoder (VAE), which creates a smooth and continuous latent space that is particularly well suited for the generative task of synthesizing new CT images \hat{x}. Because the smooth latent space of a VAE ensures that small, logical steps in the latent space correspond to small, logical changes in the output image, a VAE allows the latent diffusion model 400 to generate output CT images z with realistic and anatomically coherent transitions between CT slices. Additionally, a standard autoencoder might learn to compress and decompress the training data 220 perfectly, but the latent space could have gaps or “holes” between the latent representations z of those known images x. In those instances, the latent diffusion model 400 may generate a new latent representation \hat{z} that falls into one of these holes, in which case the decoder 360 may not have been trained how to interpret that new latent representation \hat{z} and could potentially produce a nonsensical or distorted synthesized CT image 280. By contrast, the VAE training process forces the latent space to be well-organized and continuous, which helps the decoder 360 better appreciate and interpret any novel latent representations 2 generated by the latent diffusion model 400.

In other embodiments, the autoencoder 300 may be any other type of autoencoder that is capable of being trained to map input CT images x to latent representations z that can be used to synthesize output CT images \hat{x} indicative of the input CT images x (e.g., a standard autoencoder, a denoising autoencoder, a sparse autoencoder, etc.).

In preferred embodiments, the neural network denoiser 480 may be a convolutional network for image segmentation (commonly referred to as a “U-Net”), which is particularly well-suited for the kind of image-to-image task performed by the SCOPE system 200. A U-Net architecture consists of a downsampling (encoder) path and an upsampling (decoder) path, which are linked by “skip connections.” The encoder path progressively downsamples the input, which allows the network to capture broad, contextual information (in this instance, learning the overall anatomical structure from the noisy latent representation). The decoder path progressively upsamples the data back to its original size, allowing the U-Net to reconstruct a detailed output using the context learned by the encoder to make precise, localized predictions. The most important feature of the U-Net are the skip connections that pass high-resolution feature information directly from the downsampling path to the upsampling path, allowing the neural network denoiser 480 to recover fine-grained details that would otherwise be lost during the downsampling process and ensuring the final generated images are sharp and anatomically accurate.

In other embodiments, the neural network denoiser 480 may be any suitable architecture capable of being trained to predict the noise E injected by the forward diffuser 420. The neural network denoiser 480 may be realized, for example, as a generative adversarial networks (GAN), which are known for producing very sharp images (but are often much more difficult and unstable to train than diffusion models), vision transformers (ViT), which excel at capturing long-range relationships in data (but typically require significantly more training data 220 than CNN-based models like the U-Net to achieve comparable performance), etc.

To reduce computational consumption while maintaining anatomical information, each chest CT image 224 and abdominal CT image 228 in the training data 220 (e.g., each having image dimensions, for example of 512×512 pixels) may be downsampled in the axial plane (e.g., with a Gaussian blurring as an anti-aliasing filter) to 256×256 pixels. Image intensities may be clipped (e.g., to [−1024, 3072] Hounsfield Units) and normalized (e.g., to range of [−1, 1]).

The autoencoder 300 may utilize the implementation provided in MONAI 1.2. The neural network denoiser 480 may be a U-Net with four downsampling levels. The forward diffuser 420 may employ cosine noise scheduling as recommended in the literature. The number of diffusion steps T may be 1000. The training data 220 used to train the autoencoder 300 and the latent diffusion model 400 may include chest CT images 224 from N=500 subjects in the NLST chest dataset (no lung cancer cohort) and abdominal CT images 228 from an additional 300 subjects.

The data described herein may be stored on any non-transitory computer readable storage media. Elements of the SCOPE system 200 may be realized as software instructions stored on any non-transitory computer readable storage media and executed by any suitable hardware computing device having a hardware processing unit. For example, the autoencoder 300 and the latent diffusion model 400 may be trained using an NVIDIA A6000 GPU with 48 GB of RAM. As those of ordinary skill in the art will recognize, the SCOPE system 200 may be realized as a computing device that performs the zero-shot FOV extension process 500 using an autoencoder 300 and a latent diffusion model 400 that were previously trained (using the training process 210 described above), for example by a separate computing device.

While the SCOPE system 200 is described above as being trained on chest CT images 224 and abdominal CT images 228 to extend the field of view of input chest CT images 260, those of ordinary skill the art will recognize that the SCOPE system 200 can be trained on any number of datasets (preferably datasets having overlapping regions 226) to model the complex anatomical structures and relationships and extend the field of view of any input CT images 260. Similarly, while the SCOPE system 200 is described as being trained on longitudinally-distributed axial CT images 220 to extend the longitudinal field of view of longitudinally-distributed axial input CT images 260 (by synthesizing additional axial CT images 280 that are outside the longitudinal field of view of the input CT images 260), those of ordinary skill the art will recognize that the SCOPE system 200 can be trained on any three-dimensional CT data (whether sliced along an axial plane as described above, a coronal or frontal plane, a sagittal plane) and the anatomical relationships learned by the latent diffusion model 400 can be used to generate synthesized CT images 280 that extend the field of view of any input CT images 260 in any direction.

### Experiments and Results

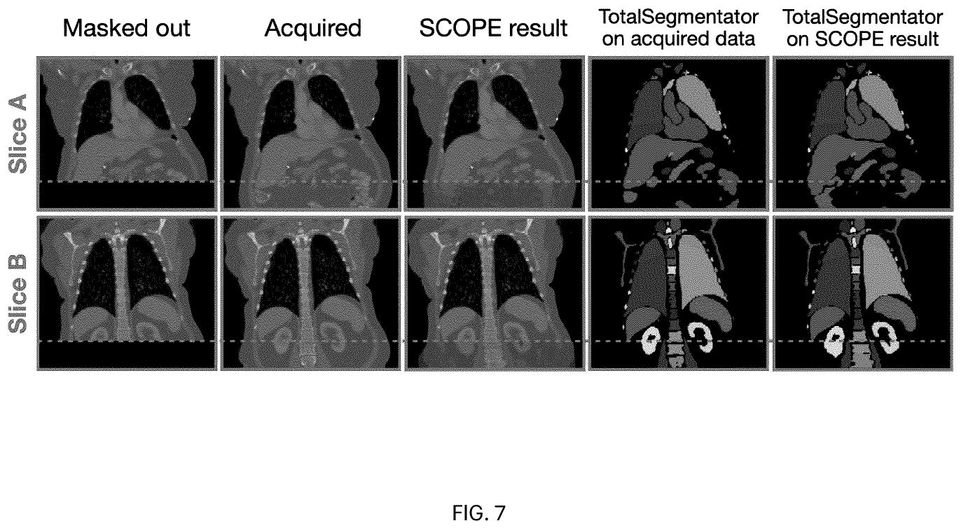

As shown in FIG. 7, we masked out the lower abdominal region of full FOV CT images to simulate the limited FOV of the NLST dataset. The SCOPE system 200 was then applied to extend the FOV on the masked-out data. The right two columns show segmentation results of TotalSegmentator on original image provided to the SCOPE system and SCOPE-extended images, respectively.

To evaluate the performance of the SCOPE system 200, we conducted both qualitative and quantitative experiments using a held-out dataset of body CT images (N=10) that cover both the chest and abdominal regions. The body CT images were preprocessed following the same procedure as described above with respect to data preprocessing. After preprocessing, we then masked-out the lower abdominal regions of the images to simulate the limited FOV of the NLST dataset. As shown in FIG. 7 left, the masked-out image has a similar FOV as the NLST data, which only partially cover the liver and the kidneys (NLST FOV shown in FIG. 1A). The SCOPE system 200 was then employed to extend the FOV by generating 30 additional axial slices (equivalent to 9 cm). The synthetic axial slices are concatenated with the provided slices to generate a 3D volume, as shown in FIG. 7 from coronal view. To further study the anatomical fidelity of the synthetic images, we ran TotalSegmentator on both the provided ground truth image and the synthetic image. FIG. 7 right shows that SCOPE not only generates realistic-looking images but also produces segmentation results that show strong agreement with the original images provided to the SCOPE system 200. A notable property of the SCOPE system 200 is that the information in the provided slices does not change during imputation. This is due to the 2D VAE design of the SCOPE system 200, which allows each slice to be processed individually while maintaining the 3D context in latent space.

TABLE 1

| # of imputed slices | 10 | 20 | 30 |
|---|---|---|---|
| SSIM(%)↑ | 81.23 ± 1.87 | 77.13 ± 2.32 | 74.14 ± 2.91 |
| PSNR(dB)↑ | 24.60 ± 0.61 | 23.55 ± 0.62 | 21.19 ± 0.72 |

To quantitatively evaluate SCOPE, we calculated the structural similarity index (SSIM) and peak signal-to-noise ratio (PSNR) given different number of imputed slices. Different number of imputed slices impacts the number of slices that can be used as conditions in Eq. 4, thus impacting the overall performance of SCOPE. As shown in Table 1, SCOPE has the best performance when the number of missing slices is 10 (equivalent to 3 cm of the human body to be imputed). As the number of imputed slices becomes larger, SCOPE has decreased performance, because it has less slices to use as condition.

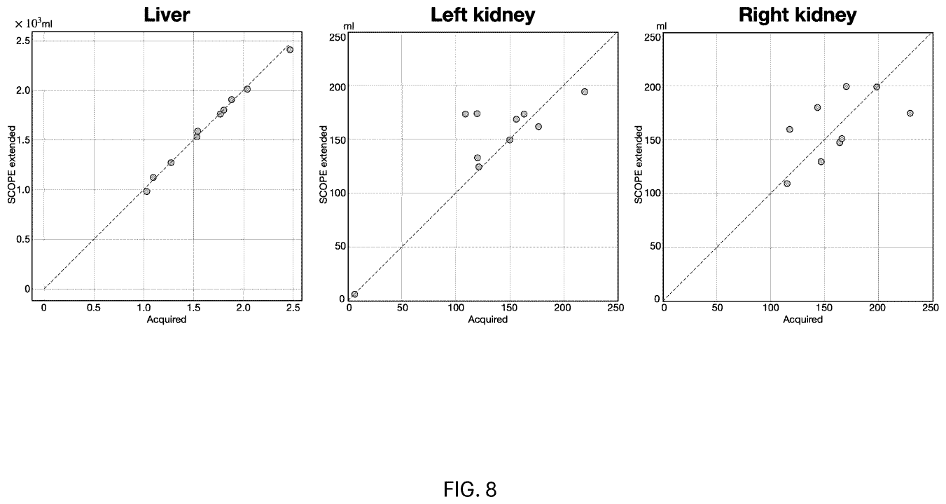

FIG. 8 are graphs illustrating volumetric agreement of the liver and kidneys between acquired ground truth images and synthetically extended images.

To further evaluate the ability of the SCOPE system 200 in generating new slices with high anatomical fidelity, we conducted a downstream task. We used TotalSegmentator to generate liver and kidney labels for both synthetic images and the original ground truth images, and we calculated the agreement between the two segmentation scenarios. As shown in FIG. 8, the liver shows a strong volume agreement between the synthesized images and the original ground truth images, which is expected as a significant proportion of the liver is included in the FOV of the original ground truth images. This provides ample contextual information for accurate FOV extension. We define the volume disagreement ratio as

$${R}_{organ} = \frac{\vert {V}_{orig} - {V}_{syn} \vert}{{V}_{orig}} \times 100 \% \qquad [ Eq . 5 ]$$

where V_{orig} denotes the organ volume of the original ground truth image and V_{syn} denotes the organ volume of the synthetic image. Over the 10 held-out subjects, R_{Liver}=1.58%±0.92%.

FIG. 8 (middle and right) shows the segmentation results of left and right kidney, which exhibit slightly reduced volume agreement. This can be attribute to their smaller size and partial coverage in the FOV of the original ground truth images. Despite these challenges, the volume disagreement ratio over the held-out data is R_{Kidney-L}=11.8%±9.2% for the left kidney and R_{Kidney-R}=12.1%±9.8% for the right kidney. An interesting observation is that there is a subject exhibiting very low volume of their left kidney. Upon detailed examination, we identified that the subject does not have a left kidney, which is a completely incidental finding.

We finally applied the SCOPE system 200 to N=100 NLST chest CT images to extend their FOV. Since the ground truth of these extended regions is unavailable, we evaluated the results implicitly. We employed the improved BPR model on SCOPE-extended images to assess whether SCOPE successfully infers and generates the missing anatomical regions. FIG. 1B shows the results of BPR on the FOV-extended images compared to the original NLST images. The shaded area 190 indicates that the SCOPE system 200 effectively extends the FOV to include regions covering the liver and kidneys.

As described in detail above, the SCOPE system 200 provides a novel method for extending the FOV in input CT images 260 using an latent diffusion model 400. By leveraging the natural overlapping regions 226 in training data 220 (e.g., chest CT images 224 and abdominal CT images 228), the SCOPE system 200 can generate anatomically consistent slices to cover regions beyond the initially acquired input CT images 260. Through qualitative and quantitative evaluations, we demonstrated that the SCOPE system 200 effectively extends the FOV to include critical regions such as the liver and the lungs. Accordingly, the SCOPE system 200 presents an advancement in the field of FOV extension and medical image synthesis. It demonstrates effectiveness in synthesizing extended anatomical regions from acquired CT images and enables the potential of providing deeper insights into the interplay between different organs of the human body.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A system for extending the field of view of computed tomography (CT) images, comprising:
an autoencoder (AE) encoder, trained on training data comprising training CT images, that generates latent representations of each of the training CT images, the latent representations being stacked to form three-dimensional stacked latent representations of the training CT images;
a latent diffusion model, trained on the three-dimensional stacked latent representations to model anatomical relationships in the three-dimensional stacked latent representations;
a hardware computer processing unit that receives input CT images having a field of view and uses the latent diffusion model to generate additional latent representations; and
an AE decoder that generates additional synthesized CT images outside the field of view of the input CT images in accordance with the additional latent representations generated using the latent diffusion model.

2. The system of claim 1, wherein the latent diffusion model is trained to model the anatomical relationships by:
injecting noise into the three-dimensional stacked latent representations during a forward diffusion process; and
training a neural network denoiser to predict the injected noise during a reverse diffusion process.

3. The system of claim 2, wherein the hardware processing unit uses the latent diffusion model to generate additional latent representations by:
generating predicted representations during each of a plurality of steps of the reverse diffusion process by using the neural network denoiser to predict the injected noise; and
guiding the reverse diffusion process by correcting the predicted representations based on latent representations of the input CT images generated by the AE encoder.

4. The system of claim 3, wherein the neural network denoiser is trained without guiding the reverse diffusion process by correcting predicted representations based on the latent representations of the training CT images.

5. The system of claim 2, wherein the neural network denoiser is a convolutional network for image segmentation.

6. The system of claim 1, wherein the autoencoder comprises a variational autoencoder (VAE).

7. The system of claim 1, wherein:
the input CT images are longitudinally-distributed axial CT slices having a field of view along a longitudinal direction; and
the additional synthesized CT images are outside the field of view of the input CT images in the longitudinal direction.

8. The system of claim 1, wherein:
the training CT images comprise a first dataset of CT images of a first anatomical region and a second dataset of CT images of a second anatomical region that partially overlaps with the first anatomical region.

9. The system of claim 8, wherein the training CT images comprise chest CT images and abdominal CT images.

10. The system of claim 9, wherein:
the input CT images comprise input chest CT images of a patient; and
the synthesized CT images comprise abdominal CT images of the patient generated based on the input chest CT images of the patient and the modeled anatomical relationships.

11. A neural network-enabled method for extending the field of view of computed tomography (CT) images, the method comprising:
encoding training CT images, by an autoencoder (AE) encoder trained on training data comprising the training CT images, to form latent representations of each of the training CT images;
stacking the latent representations to form three-dimensional stacked latent representations of the training CT images;
receiving input CT images having a field of view;
encoding the input CT images to form latent representations of each of the input CT images;
using a latent diffusion model, trained on the three-dimensional stacked latent representations to model anatomical relationships in the three-dimensional stacked latent representations, to generate additional latent representations; and
generating additional synthesized CT images outside the field of view of the input CT images, by an AE decoder trained on the training data, in accordance with the additional latent representations generated using the latent diffusion model.

12. The method of claim 11, wherein the latent diffusion model is trained to model the anatomical relationships by:
injecting noise into the three-dimensional stacked latent representations during a forward diffusion process; and
training a neural network denoiser to predict the injected noise during a reverse diffusion process.

13. The method of claim 12, wherein the additional latent representations are generated by performing a guided reverse diffusion process comprising:
generating predicted representations during each of a plurality of steps of the reverse diffusion process by using the neural network denoiser to predict the injected noise; and
guiding the reverse diffusion process by correcting the predicted representations based on the latent representations of the input CT images.

14. The method of claim 13, wherein the neural network denoiser is trained without guiding the reverse diffusion process by correcting predicted representations based on the latent representations of the training CT images.

15. The method of claim 12, wherein the neural network denoiser is a convolutional network for image segmentation.

16. The method of claim 11, wherein the autoencoder comprises a variational autoencoder (VAE).

17. The method of claim 11, wherein:
the input CT images are longitudinally-distributed axial CT slices having a field of view along a longitudinal direction; and
the additional synthesized CT images are outside the field of view of the input CT images in the longitudinal direction.

18. The method of claim 11, wherein:
the training CT images comprise a first dataset of CT images of a first anatomical region and a second dataset of CT images of a second anatomical region that partially overlaps with the first anatomical region.

19. The method of claim 18, wherein the training CT images comprise chest CT images and abdominal CT images.

20. The system of claim 19, wherein:
the input CT images comprise input chest CT images of a patient; and
the additional synthesized CT images comprise abdominal CT images of the patient generated based on the input chest CT images of the patient and the modeled anatomical relationships.
